(19) United States
(12) Patent Application Publication
(10) Pub. No.: US 2015/0004120 A1
(43) Pub. Date: Jan. 1, 2015

(54) COMPOSITIONS AND METHODS FOR TREATING HAIR

(71) Applicant: L'OREAL, Paris (FR)

(72) Inventors: Siliu TAN, Westfield, NJ (US); Nghi Van NGUYEN, Edison, NJ (US); Jim SINGER, South Orange, NJ (US); Jean-Thierry SIMONNET, Mamaroneck, NY (US)

(21) Appl. No.: 13/931,248

(22) Filed: Jun. 28, 2013

(51) Int. Cl. A61K 8/87; A61K 8/81; A45D 7/06; A61Q 5/06

(52) U.S. Cl. CPC A61K 8/87; A61Q 5/06; A61K 8/8147; A45D 7/06; A45D 2007/002
USPC 424/70.16; 132/206

(57) ABSTRACT

Disclosed are hair styling compositions comprising a combination of hybrid and simple latex polymers, wherein at least one simple latex polymer is a film-forming polymer. The composition is capable of forming a film having certain properties on a substrate. Methods of styling the hair are also disclosed.

### TECHNICAL FIELD

The disclosure relates to hair styling compositions comprising a combination of hybrid and simple latex polymers, wherein at least one simple latex polymer is a film-forming polymer. In various embodiments of the disclosure, the hybrid and simple latex polymers are chosen to have certain properties. Compositions comprising the hybrid and simple latex polymers may, according to certain embodiments, form films that have surprising properties. Methods of styling the hair with such compositions are also disclosed.

### BACKGROUND

Compositions for styling the hair are known, such as, for example, hair spray compositions, hair gels and mousses, hair volumizing compositions, hair smoothing creams, lotions, serums, oils, clays, etc. The goals of many hair styling compositions include to hold or fix the hair in a particular shape, to impart or increase volume of the hair, and/or to smooth the hair, e.g. to decrease or eliminate the appearance of frizz.

Drawbacks associated with current products for styling the hair include that the product is often sticky or tacky and/or often produces a film that imparts a sticky or tacky feel, and styled hair that is stiff and/or “crunchy” (i.e. the film is hard and brittle resulting in a crunching feel or sound when the hair is touched), which is undesirable for most consumers.

Current products for styling the hair typically include water soluble film-forming polymers. Depending on the chemical make-up of these polymers, they may be either soluble in water, or they may be water insoluble polymers which are made water soluble via various chemical modifications, such as neutralization. Solutions comprising these polymers tend to be viscous, i.e. as the concentration of the polymer increases, its viscosity builds up rapidly. Translated to styling applications, as the solvent evaporates, the polymer solution becomes thicker on the hair surface, resulting in a sticky or tacky film. These products also tend to exhibit problems with product spreadability, hair manageability, and low degree of humidity resistance which is particularly a problem in hot and humid countries.

The use of latex polymers is also known, for example, to provide extended-wear properties to a cosmetic product (e.g. mascara, eyeliner, nail polish) into which they are formulated.

Some known compositions include one latex polymer. For example, U.S. Pat. No. 6,126,929 describes a composition comprising a dispersion of a latex film former, optionally with a plasticizer, and a non film-forming particle not capable of being film-formed. U.S. Pat. No. 4,710,374 describes a composition comprising cationic polymers, a surfactant, and an anionic latex. U.S. Pat. No. 7,740,832 describes a composition comprising at least one non-latex polymer and an anionic, cationic or amphoteric fixing polymer. U.S. Pat. No. 4,798,721 describes a composition comprising a latex particle. U.S. Patent Application No. 2005/0089490 A1 describes a composition comprising a water-dispersible styling polymer and a gel-forming polymer.

Other known cosmetic compositions include various components to provide improved properties such as adhesion, flexibility, and compatibility of other components. For example, U.S. Patent Application No. 2007/0224140 A1 describes a composition comprising a cosmetically acceptable medium, a non film-forming microsphere to provide adhesion, and a film-forming component comprising two water-borne emulsion polymers. French Patent Application No. FR 2 968 978A describes an eyeliner composition comprising at least two film-forming latexes and a plasticizer to increase the flexibility of the film. French Patent Application No. FR 2 898 050A describes a composition comprising a fatty acid ester, and a copolymer of a (meth)acrylate polymer and a hydroxyester (meth)acrylate. U.S. Patent Application No. 2009/0297467A describes a composition comprising at least one neutralized sulfonated polymer and mixtures of acrylates and hydroxyester acrylates. U.S. Patent Application No. 2009/035335 A1 describes a mascara composition comprising two water-dispersible acrylate polymers, and a cross-linked polymeric film-former to enhance the compatibility and bind the two water-dispersible acrylate polymers. International Patent Application No. WO 2011/137338 A2 describes a composition comprising a polyurethane dispersion and an acrylic film-forming dispersion. U.S. Patent Application No. 2004/0071646A describes an aerosol device containing a composition comprising a polyurethane dispersion having a particle size of from 0.1-1 μm, and at least one non-latex fixing polymer.

Cosmetic compositions in a non-aqueous medium are known. For example, European Patent Application No. EP 1 082 953A describes a dispersion comprising two film formers in isododecane. International Patent Application No. WO11056332A describes a composition comprising three volatile solvents, and at least one film former, for example silicon acrylate or acrylate, soluble or dispersible in at least one of the three solvents.

Compositions for use in mascaras may have low glass transition temperatures (“Tg”) to obtain a soft film. For example, U.S. Patent Application No. 2010/0028284 A1 describes a mascara composition comprising at least two acrylate film formers, where the glass transition temperature (“Tg”) of the mascara composition is 20° C. U.S. Patent Application No. 2006/134043A describes a mascara composition comprising a fatty acid and at least one acrylate resin emulsion.

Some known compositions use solubilized polymers rather than polymer particles. For example, U.S. Pat. No. 7,651,693 describes a composition comprising a solubilized blend of two polymers. U.S. Pat. No. 6,214,328 describes a composition comprising at least one acrylate latex that is soluble in solutions containing low volatile organic compounds or in water upon neutralization.

U.S. Pat. No. 5,441,728 describes a composition comprising a water-soluble fixative polymer and a latex particle. Water-soluble polymers tend to be sticky, and may not be suitable for applications requiring a clean touch.

French Patent Application No. FR 2 834 458A describes a nail polish composition comprising two film formers in an aqueous medium in a specific ratio.

Additionally, attempts have been made to make latex particles in which a soft polymer is combined with a hard polymer, co-existing in the same particle. These attempts include core shell latexes, wherein a hard internal polymer is surrounded by a soft external polymer, and interpenetrating network polymers (IPN), wherein the soft polymer is interwoven with the hard polymer.

For example, U.S. Patent Application No. 2003/0064045 A1 describes a mascara composition comprising a dispersion of particles having a core-shell structure. U.S. Patent Application No. 2007/0286833 A1 describes a multistage polymer comprising a latex core-shell particle comprising a soft polymer and a hard polymer. In addition, U.S. Patent Application No. 2009/0317432 A describes an applicator for makeup containing a composition comprising a colorant and at least one latex or core-shell latex particle.

Further, U.S. Patent Application Nos. 2003/053976 A and 2003/044440 A describe cosmetic make-up or care compositions comprising core shell latexes. U.S. Patent Application No. 2009/0297467 describes a hair styling composition that comprises a core shell latex and a non-latex solid sulfonated polymer.

However, it has now been discovered that by providing a composition comprising a combination of hybrid and simple latex polymers, wherein at least one of said latex polymers is a film-forming polymer, and wherein each latex polymer is selected to have particular properties, it is possible to form a film on a substrate that has certain desirable properties, such as a clean, natural, and/or “invisible” feel, and a lack of stickiness. Such compositions may be useful in hair-styling applications wherein styling benefits such as a natural look, curling or straightening, and styling hold are imparted to the hair.

Moreover, compositions according to embodiments of the disclosure may be prepared that deliver a surprisingly broad range of hair styling benefits, such as, for example, from low to high style-hold and curl-retention properties, for example by varying the weight ratio between both latex polymers, with or without additives.

### DESCRIPTION OF EXEMPLARY EMBODIMENTS

The disclosure relates, in various embodiments, to compositions comprising a combination of at least one hybrid latex polymer and at least one simple latex polymer, wherein the at least one simple latex polymer is a film-forming polymer. In further embodiments, the disclosure relates to compositions comprising a combination of at least one hybrid latex polymer and at least two simple latex polymers, wherein at least one simple latex polymer is a film-forming polymer.

In various embodiments, the latex polymers may be chosen to have certain properties. In at least certain embodiments, the at least one hybrid latex polymer is present in an amount ranging from about 0.1% to about 90%, relative to the weight of the composition, and the at least one simple latex polymer is present in an amount ranging from about 0.1% to about 30% by weight, relative to the weight of the composition. In embodiments where the composition comprises at least two simple latex polymers, the at least two simple latex polymers are present in the composition in a weight ratio of about 10:1 to about 1:10.

The composition comprising the combination of hybrid and simple polymers forms a film when applied to a substrate. In at least certain exemplary embodiments according to the disclosure, the resulting film formed by the composition comprising at least one simple latex polymer and at least one latex hybrid polymer is clear and/or transparent.

In further embodiments, methods of styling the hair are disclosed, said methods comprising applying compositions according to the disclosure to the hair. Such styling methods may comprise shaping, reshaping, positioning, repositioning, adding volume to, curling, or straightening the hair, in order to achieve a certain hair style or appearance.

### Latex Polymers

### Simple Latexes

According to various exemplary embodiments of the disclosure, the compositions comprise at least one simple latex polymer, wherein said at least one simple latex polymer is a film-forming polymer. In further exemplary embodiments, the compositions comprise at least two simple latex polymers, at least one of which is a film-forming polymer. As used herein, the phrase “simple latex polymer” is meant to include a latex polymer other than the hybrid latex polymer of the present disclosure.

The simple latex polymers may be chosen to provide a composition that produces a film when applied to a substrate, wherein the film has a Young's modulus from about 0.1 MPa to about 6 GPa, and/or a strain, under stress at 0.5 MPa, that ranges up to about 300%.

In various exemplary embodiments, the at least one simple latex polymer is present in an amount ranging from about 0.1% to about 30% by weight, relative to the weight of the composition. In at least certain exemplary embodiments, the at least one simple latex polymer is chosen from polymers that may be identified as polymer A and polymer B. Compositions according to certain embodiments may comprise at least one polymer A or at least one polymer B, wherein the at least one polymer A or at least one polymer B is a film-forming polymer.

In further exemplary embodiments of the disclosure, the compositions comprise at least two simple latex polymers, wherein the simple latex polymers are present in a combined amount ranging from about 0.1% to about 30% by weight, relative to the weight of the composition. In other embodiments, the at least two simple latex polymers are present in the composition in a weight ratio of about 10:1 to about 1:10, such as about 4:1 to about 1:4.

In various exemplary and non-limiting embodiments, the compositions comprise at least two simple latex polymers, at least one of which is a film-forming polymer, wherein the at least two simple latex polymers are chosen from polymer A and polymer B. Compositions according to certain embodiments may, by way of example, comprise at least one polymer A and at least one polymer B, at least two polymers A, or at least two polymers B, wherein at least one of polymer A and/or polymer B is a film-forming polymer.

In various embodiments, polymer A may be chosen from latex polymers having a Young's modulus ranging from about 0.1 MPa to about 10 MPa, and a strain, under stress at 0.5 MPa, of at least about 1%; and polymer B may be chosen from latex polymers having a Young's modulus ranging from about 10 MPa to about 6 GPa, and a strain, under stress at 0.5 MPa, of less than about 5%. In at least certain embodiments, polymer A may have a glass transition temperature (Tg) ranging from about −90° C. to about 40° C., and polymer B may have a glass transition temperature (Tg) ranging from about 40° C. to about 200° C. In at least certain other embodiments, the weight ratio of polymer A to polymer B in the compositions of the disclosure is from about 1:10 to about 1:1, from about 3:1 to about 10:1, or from about 5:1 to about 10:1.

In at least certain exemplary and non-limiting embodiments, simple latex polymers A and B may be chosen such that polymer A comprises at least one latex polymer which is optionally a film-forming polymer that is a relatively soft, flexible latex polymer, and polymer B comprises at least one latex polymer which is optionally a film-forming polymer that is a relatively hard, brittle polymer, although such characteristics are not required.

At least one of polymer A and polymer B is a film-forming polymer. In various exemplary embodiments, latex polymer A is a film-forming polymer and latex polymer B is a non-film-forming polymer. In further exemplary embodiments, latex polymer A is a non-film-forming polymer and latex polymer B is a film-forming polymer. In yet further exemplary embodiments, latex polymer A is a film-forming polymer and latex polymer B is a film-forming polymer.

As used herein, a film-forming polymer is meant to include a polymer that is capable, by itself or in the presence of an auxiliary film-forming agent, of forming a macroscopically continuous film that adheres to keratin materials, and preferably a cohesive film, better still, a film whose cohesion and mechanical properties are such that said film can be isolated and manipulated individually, for example, when said film is prepared by pouring onto a non-stick surface such as Teflon-coated or silicone-coated surface. In addition, as used herein, a non-film-forming polymer is meant to include a polymer which will not form a film at ambient temperature or below, or in other words, will only form a film at temperatures above ambient. For purposes of this disclosure, ambient temperature is taken as being below 40° C. such as in the range of 15° C. to 30° C.

By “at least one simple latex polymer” and “at least two simple latex polymers,” it is contemplated that more than one or more than two simple latex polymers may be chosen. Thus, for example, in various embodiments, both polymers A and B in the composition of the disclosure may be simple latex film-forming polymers, and the composition may also comprise at least one simple latex polymer that is a non-film-forming polymer; or one of polymer A and B may be a film-forming polymer while the other is a non-film-forming polymer, but at least one additional film-forming (latex or non-latex) polymer may also be added; and so on.

In further embodiments, the composition comprises exactly one simple latex polymer, which is a film forming polymer. In further embodiments, the composition comprises at least two simple latex polymers, at least one of which is a film-forming polymer. In yet further embodiments, the composition comprises at least one or at least two simple latex polymers, one or both of which are film-forming polymers, but does not comprise any additional film-forming simple latex polymers.

In at least certain embodiments of the disclosure, the simple latex polymers may be provided in the form of aqueous dispersions prior to formulating the compositions of the disclosure. In various embodiments, the aqueous dispersions may be obtained through an emulsion polymerization of monomers wherein the resulting latex polymers have a particle size lower than about 1 μm. In at least one exemplary embodiment, a dispersion prepared by the polymerization in water of one or more monomers having a polymerizable double bond may be chosen. In another exemplary embodiment, the aqueous dispersions obtained through an emulsion polymerization may be spray-dried.

In other embodiments, the simple latex polymers are produced from condensation reactions between monomers and subsequently dispersed in an aqueous medium.

Thus, the simple latex polymers may, in various exemplary embodiments, exist as dispersed polymer particles in a dispersion medium, such as an aqueous dispersion medium. The simple latex polymers may, in certain embodiments, each be dispersed in independent dispersion media. In yet further embodiments, the simple latex polymers may be dispersed together in the same dispersion medium.

The dispersion medium comprises at least one solvent chosen from water. The dispersion medium may further comprise at least one solvent chosen from cosmetically acceptable organic solvents. Cosmetically acceptable organic solvents may, in various embodiments, be water-miscible, e.g. capable of forming at 25° C. a homogeneous mixture that is transparent, or substantially transparent, to the eye. For instance, cosmetically acceptable organic solvents may be chosen from lower monoalcohols, such as those containing from about 1 to 5 carbon atoms, for example ethanol and isopropanol; polyols, including glycols, such as those containing from about 2 to 8 carbon atoms, for example propylene glycol, ethylene glycol, 1,3-butylene glycol, dipropylene glycol, hexylene glycol, and glycerin; hydrocarbons, such as, for example, isododecane and mineral oil; and silicones, such as dimethicones, cyclomethicones, and cyclopentasiloxane; as well as mixtures thereof.

In at least one embodiment, the solvent of the dispersion medium consists of water. In other embodiments, the solvent of the dispersion medium consists of water and at least one cosmetically acceptable organic solvent. In further embodiments, the solvent comprises water. In yet further embodiments, the solvent of the dispersion medium primarily comprises water. For example, the solvent of the dispersion medium may, in at least certain exemplary embodiments, comprise greater than 50% water, such as greater than 55% water, greater than 60% water, greater than 65% water, greater than 70% water, greater than 75% water, greater than 80% water, greater than 85% water, greater than 90% water, greater than 95% water, greater than 96% water, greater than 97% water, greater than 98% water, or greater than 99% water.

In embodiments according to the disclosure, the simple latex polymer particles are not soluble in the solvent of the dispersion medium, i.e. are not water soluble and/or are not soluble in the at least one cosmetically acceptable organic solvent. Accordingly, the simple latex polymers retain their particulate form in the solvent or solvents chosen.

In at least certain exemplary embodiments, the simple latex particles according to the disclosure may have an average diameter ranging up to about 1000 nm, such as from about 50 nm to about 800 nm, or from about 100 nm to about 500 nm. Such particle sizes may be measured with a laser granulometer (e.g. Brookhaven BI90).

In various embodiments, the simple latex polymers may, independently, be neutralized, partially neutralized, or unneutralized. In exemplary embodiments where the simple latex polymers are neutralized or partially neutralized, the particle size may be, for example, greater than about 800 nm. In at least certain embodiments, the particulate form of the simple latex polymers is retained in the dispersion medium.

In further embodiments, the simple latex polymers may be chosen from uncharged and charged latex polymers. Thus, the simple latex polymers may, according to various exemplary embodiments, be chosen from nonionic latex polymers, cationic latex polymers, and anionic latex polymers.

As non-limiting examples of simple latex polymers that may be used, mention may be made, independently, of simple acrylate latex polymers and simple polyurethane latex polymers. When two simple latex polymers are used, one of the simple latex polymers can be chosen from simple acrylate latex polymers and the other can be chosen from simple polyurethane latex polymers; or both can be chosen from simple polyurethane latex polymers; or both can be chosen from simple acrylate latex polymers.

By way of non-limiting example only, the simple latex polymers may be chosen from simple acrylate latex polymers, such as those resulting from the homopolymerization or copolymerization of monomers chosen from (meth)acrylics, (meth)acrylates, (meth)acrylamides and/or vinyl homopolymers or copolymers. The term “(meth)acryl” and variations thereof, as used herein, means acryl or methacryl.

The (meth)acrylic monomers may be chosen from, for example, acrylic acid, methacrylic acid, citraconic acid, itaconic acid, maleic acid, fumaric acid, crotonic acid, and maleic anhydride. Additional non-limiting examples of (meth)acrylic monomers include C1-C8 alkyl (meth)acrylic, such as, for example, methyl (meth)acrylic, ethyl (meth)acrylic, propyl (meth)acrylic, isopropyl (meth)acrylic, butyl (meth)acrylic, tert-butyl (meth)acrylic, pentyl(meth) acrylic, isopentyl (meth)acrylic, neopentyl (meth)acrylic, hexyl (meth)acrylic, isohexyl (meth)acrylic, 2-ethylhexyl (meth)acrylic, cyclohexyl (meth)acrylic, isohexyl (meth)acrylic, heptyl (meth)acrylic, isoheptyl (meth)acrylic, octyl (meth)acrylic, isooctyl (meth)acrylic, as well as combinations of any of the above.

The esters of (meth)acrylic monomers may be, by way of non-limiting example, C1-C8 alkyl (meth)acrylates such as methyl (meth)acrylate, ethyl (meth)acrylate, propyl (meth)acrylate, isopropyl (meth)acrylate, butyl (meth)acrylate, tert-butyl (meth)acrylate, pentyl(meth) acrylate, isopentyl (meth)acrylate, neopentyl (meth)acrylate, hexyl (meth)acrylate, isohexyl (meth)acrylate, 2-ethylhexyl (meth)acrylate, cyclohexyl (meth)acrylate, isohexyl (meth)acrylate, heptyl (meth)acrylate, isoheptyl (meth)acrylate, octyl (meth)acrylate, isooctyl (meth)acrylate, allyl (meth)acrylate, and combinations thereof. Additional and non-limiting examples include C1-C8 alkoxy (meth)acrylates, such as methoxy (meth)acrylate, ethoxy (meth)acrylate, propyl oxide (meth)acrylate, isopropyl oxide (meth)acrylate, butyl oxide (meth)acrylate, tert-butyl oxide (meth)acrylate, pentyl oxide (meth) acrylate, isopentyl oxide (meth)acrylate, neopentyl oxide (meth)acrylate. The esters may be, by way of non-limiting example, C2-C6 hydroxy alkyl (meth)acrylates, such as hydroxy ethyl (meth)acrylate, 2-hydroxypropyl (meth)acrylate, glycidyl (meth)acrylate, ethylene glycol di(meth)acrylate, polyethylene glycol mono(meth)acrylate, 1,4-butane diol di(meth)acrylate, 1,6,hexane diol di(meth)acrylate, and any combination thereof. The esters may be, by way of non-limiting example, aryl (meth)acrylates such as benzyl (meth)acrylate, phenyl (meth)acrylate, and any combination thereof. The esters can further contain amino groups such as aminoethyl (meth)acrylate, N,N-dimethylaminoethyl (meth)acrylate, N,N-dimethylaminopropyl (meth)acrylate, N,N-dimethylaminodimethylpropyl (meth)acrylate, N,N-diethyleaminoethyl (meth)acrylate, and N,N,N-trimethylaminoethyl (meth)acrylate; and salts of the ethylenic amines.

According to at least certain exemplary embodiments, the alkyl group of the esters may be either fluorinated or perfluorinated, e.g. some or all of the hydrogen atoms of the alkyl group are substituted with fluorine atoms. The monomers can also be fluorine-containing monomers, such as, by way of non-limiting example, trifluoroethyl methacrylate, 2,2,3,3-tetrafluoropropyl methacrylate, 2,2,3,3,4,4-hexafluorobutyl methacrylate, perfluorooctyl methacrylate and perfluorooctyl acrylate; and silicone macromonomers.

The amides of (meth)acrylic monomers can, for example, be made of (meth)acrylamides, and especially N-alkyl (meth)acrylamides, in particular N—(C1-C12) alkyl (meth)acrylates such as N-ethyl (meth)acrylamide, N-t-butyl (meth)acrylamide, N-t-octyl (meth)acrylamide, N-methylol (meth)acrylamide and N-diacetone (meth)acrylamide, and any combination thereof.

The vinyl monomers can include, but are not limited to, vinyl cyanide compounds such as acrylonitrile and methacrylonitrile; vinyl esters such as vinyl formate, vinyl acetate, vinyl propionate, vinyl neodecanoate, vinyl pivalate, vinyl benzoate and vinyl t-butyl benzoate, triallyl cyanurate; vinyl halides such as vinyl chloride and vinylidene chloride; aromatic mono- or divinyl compounds such as styrene, α-methylstyrene, chlorostyrene, alkylstyrene, divinylbenzene and diallyl phthalate, and combination thereof. Other non-limiting ionic monomers can include para-styrensulfonic, vinylsulfonic, 2-(meth)acryloyloxyethylsulfonic, 2-(meth)acrylamido-2-methylpropylsulfonic acids.

The list of monomers given is not limiting, and it should be understood that it is possible to use any monomer known to those skilled in the art which includes acrylic and/or vinyl monomers (including monomers modified with a silicone chain).

Silicone acrylic polymers may also optionally be used as vinyl polymer in at least one exemplary and non-limiting embodiment.

In at least certain, non-limiting exemplary embodiments, acrylic latex polymers may be chosen from aqueous dispersions of Methacrylic Acid/Ethyl Acrylate copolymer (INCI: Acrylates Copolymer, such as Luviflex® Soft by BASF), PEG/PPG-23/6 Dimethicone Citraconate/C10-30 Alkyl PEG-25 Methacrylate/Acrylic Acid/Methacrylic Acid/Ethyl Acrylate/Trimethylolpropane PEG-15 Triacrylate copolymer (INCI: Polyacrylate-2 Crosspolymer, such as Fixate Superhold™ by Lubrizol), Styrene/Acrylic copolymer (such as Neocryl® A-1120, DSM), Ethylhexyl Acrylate/Methyl Methacrylate/Butyl Acrylate/Acrylic Acid/Methacrylic Acid copolymer (INCI: Acrylates/Ethylhexyl Acrylate Copolymer, such as Daitosol 5000SJ, Daito Kasei Kogyo), Acrylic/Acrylates Copolymer (INCI name: Acrylates Copolymer, such as Daitosol 5000AD, Daito Kasei Kogyo), and Acrylic copolymers and Acrylates Copolymers, such as those known under the tradenames VINYSOL 2140 (Daido Chemical), ACULYN™ 33 (Dow Chemical), LUVIMER® MAE (BASF), or BALANCE CR (AKZO NOBEL).

In yet further exemplary and non-limiting embodiments, the simple latex polymers may be chosen from simple polyurethane latex polymers, such as aqueous polyurethane dispersions comprising the reaction products of (i), (ii), and/or (iii), defined below.

Reaction product (i) may be any prepolymer according to the formula:

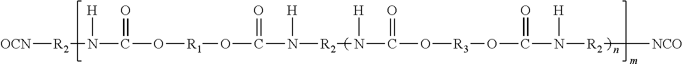

wherein R1 is chosen from bivalent radicals of a dihydroxyl functional compound, R2 is chosen from hydrocarbon radicals of an aliphatic or cycloaliphatic polyisocyanate, and R3 is chosen from radicals of a low molecular weight diol, optionally substituted with ionic groups, n ranges from about 0 to about 5, and m is greater than about 1.

Suitable dihydroxyl compounds for providing the bivalent radical R1 include those having at least two hydroxy groups, and having number average molecular weights ranging from about 700 to about 16,000, such as, for example, from about 750 to about 5000. Non-limiting examples of the high molecular weight compounds include polyester polyols, polyether polyols, polyhydroxy polycarbonates, polyhydroxy polyacetals, polyhydroxy polyacrylates, polyhydroxy polyester amides, polyhydroxy polyalkadienes and polyhydroxy polythioethers. In various embodiments, polyester polyols, polyether polyols, and polyhydroxy polycarbonates may be chosen. Mixtures of such compounds are also within the scope of the disclosure.

The polyester diol(s) may optionally be prepared from aliphatic, cycloaliphatic, or aromatic dicarboxylic or polycarboxylic acids, or anhydrides thereof; and dihydric alcohols such as diols chosen from aliphatic, alicyclic, or aromatic diols.

The aliphatic dicarboxylic or polycarboxylic acids may be chosen from, for example: succinic, fumaric, glutaric, 2,2-dimethylglutaric, adipic, itaconic, pimelic, suberic, azelaic, sebacic, maleic, malonic, 2,2-dimethylmalonic, nonanedicarboxylic, decanedicarboxylic, dodecanedioic, 1,3-cyclohexanedicarboxylic, 1,4-cyclohexanedicarboxylic, 2,5-norboranedicarboxylic, diglycolic, thiodipropionic, 2,5-naphthalenedicarboxylic, 2,6-naphthalenedicarboxylic, phthalic, terephthalic, isophthalic, oxanic, o-phthalic, tetrahydrophthalic, hexahydrophthalic or trimellitic acid.

The acid anhydrides may, in further exemplary embodiments, be chosen from o-phthalic, trimellitic or succinic acid anhydride or a mixture thereof. By way of non-limiting example only, the dicarboxylic acid may be adipic acid.

The dihydric alcohols may be chosen from, for example, ethanediol, ethylene glycol, diethylene glycol, triethylene glycol, trimethylene glycol, tetraethylene glycol, 1,2-propanediol, dipropylene glycol, tripropylene glycol, tetrapropylene glycol, 1,3-propanediol, 1,4-butanediol, 1,3-butanediol, 2,3-butanediol, 1,5-pentanediol, 1,6-hexanediol, 2,2-dimethyl-1,3-propanediol, 1,4-dihydroxycyclohexane, 1,4-dimethylolcyclohexane, cyclohexanedimethanol, 1,8-octanediol, 1,10-decanediol, 1,12-dodecanediol, neopentyl glycol, and mixtures thereof. The cycloaliphatic and/or aromatic dihydroxyl compounds may also be suitable as the dihydric alcohol(s) for the preparation of the polyester polyol(s).

The polyester diols may also be chosen from homopolymers or copolymers of lactones, which are, in at least certain embodiments, obtained by addition reactions of lactones or lactone mixtures, such as butyrolactone, c-caprolactone and/or methyl-c-caprolactone with the appropriate polyfunctional, e.g. difunctional, starter molecules such as, for example, the dihydric alcohols mentioned above. The corresponding polymers of ε-caprolactone may be chosen in at least some embodiments.

The polyester polyol, e.g. polyester diol, radical R1, may be obtained by polycondensation of dicarboxylic acids, such as adipic acid, with polyols, e.g. diols, such as hexanediol, neopentyl glycol, and mixtures thereof.

The polycarbonates containing hydroxyl groups comprise those known per se, such as the products obtained by reacting diols, such as (1,3)-propanediol, (1,4)-butanediol and/or (1,6)-hexanediol, diethylene glycol, triethylene glycol, or tetraethylene glycol with diaryl carbonates, for example diphenyl carbonate or phosgene.

Optional polyether polyols may be obtained in any known manner by reacting starting compounds which contain reactive hydrogen atoms with alkylene oxides, such as, for example, ethylene oxide; propylene oxide; butylene oxide; styrene oxide; tetrahydrofuran; or epichlorohydrin, or with mixtures of these alkylene oxides. In at least certain embodiments, the polyethers do not contain more than about 10% by weight of ethylene oxide units. For example, polyethers obtained without addition of ethylene oxide may be chosen.

Polyethers modified with vinyl polymers are also suitable according to various embodiments of the disclosure. Products of this type can be obtained by polymerization, for example, of styrene and acrylonitrile in the presence of polyethers, for example as described in U.S. Pat. Nos. 3,383,351; 3,304,273; 3,523,095; 3,110,695; and German patent 1 152 536.

Among the polythioethers which may be chosen include the condensation products obtained from thiodiglycol per se and/or with other glycols, dicarboxylic acids, formaldehyde, aminocarboxylic acids, and/or amino alcohols. The products obtained are either mixed polythioethers, polythioether esters, or polythioether ester amides, depending on the co-components.

Optional polyacetals include but are not limited to the compounds which can be prepared from aldehydes, for example formaldehyde, and from glycols, such as diethylene glycol, triethylene glycol, ethoxylated 4,4′-(dihydroxy)diphenyl-dimethylmethane, and (1,6)-hexanediol. Polyacetals useful according to various non-limiting embodiments of the disclosure can also be prepared by polymerization of cyclic acetals.

Optional polyhydroxy polyesteramides and polyamines include, for example, the mainly linear condensation products obtained from saturated or unsaturated, polybasic carboxylic acids or anhydrides thereof, and from saturated or unsaturated, polyvalent amino alcohols, from diamines, or from polyamines, as well as mixtures thereof.

Optional monomers for the production of polyacrylates having hydroxyl functionality comprise acrylic acid, methacrylic acid, crotonic acid, maleic anhydride, 2-hydroxyethyl acrylate, 2-hydroxyethyl methacrylate, 2-hydroxypropyl acrylate, 2-hydroxypropyl methacrylate, 3-hydroxypropyl acrylate, 3-hydroxypropyl methacrylate, glycidyl acrylate, glycidyl methacrylate, 2-isocyanatoethyl acrylate, and 2-isocyanatoethyl methacrylate.

Mixtures of dihydroxy compounds can also be chosen.

Optional polyisocyanates for providing the hydrocarbon-based radical R2 include, for example, organic diisocyanates having a molecular weight ranging from about 100 to about 1500, such as about 112 to about 1000, or about 140 to about 400.

Optional diisocyanates are those chosen from the general formula R₂(NCO)₂, in which R₂ represents a divalent aliphatic hydrocarbon group comprising from about 4 to 18 carbon atoms, a divalent cycloaliphatic hydrocarbon group comprising from about 5 to 15 carbon atoms, a divalent araliphatic hydrocarbon group comprising from about 7 to 15 carbon atoms, or a divalent aromatic hydrocarbon group comprising from about 6 to 15 carbon atoms. Examples of the organic diisocyanates which may be chosen include, but are not limited to, tetramethylene diisocyanate, 1,6-hexamethylene diisocyanate, dodecamethylene diisocyanate, cyclohexane-1,3-diisocyanate and cyclohexane-1,4-diisocyanate, 1-isocyanato-3-isocyanatomethyl-3,5,5-trimethylcyclohexane (isophorone diisocyanate or IPDI), bis(4-isocyanatocyclohexyl)-methane, 1,3-bis(isocyanatomethyl)cyclohexane and 1,4-bis(isocyanatomethyl)cyclohexane and bis(4-isocyanato-3-methylcyclohexyl)methane. Mixtures of diisocyanates can also be used.

In at least certain embodiments, diisocyanates are chosen from aliphatic and cycloaliphatic diisocyanates. For example, 1,6-hexamethylene diisocyanate, isophorone diisocyanate, and dicyclohexylmethane diisocyanate, as well as mixtures thereof may be chosen.

The use of diols, for example low molecular weight diols, R3, may in at least certain embodiments allow a stiffening of the polymer chain. The expression “low molecular weight diols” means diols having a molecular weight ranging from about 50 to about 800, such as about 60 to 700, or about 62 to 200. They may, in various embodiments, contain aliphatic, alicyclic, or aromatic groups. In certain exemplary embodiments, the compounds contain only aliphatic groups. The diols that may be chosen may optionally have up to about 20 carbon atoms, and may be chosen, for example, from ethylene glycol, diethylene glycol, propane-1,2-diol, propane-1,3-diol, butane-1,4-diol, 1,3-butylene glycol, neopentyl glycol, butylethylpropanediol, cyclohexanediol, 1,4-cyclohexanedimethanol, hexane-1,6-diol, bisphenol A (2,2-bis(4-hydroxyphenyl)propane), hydrogenated bisphenol A (2,2-bis(4-hydroxycyclohexyl)-propane), and mixtures thereof. For example, R3 may be derived from neopentyl glycol.

Optionally, the low molecular weight diols may contain ionic or potentially ionic groups. Suitable low molecular weight diols containing ionic or potentially ionic groups may be chosen from those disclosed in U.S. Pat. No. 3,412,054. In various embodiments, compounds may be chosen from dimethylol-butanoic acid (DMBA), dimethylolpropionic acid (DMPA), and carboxyl-containing caprolactone polyester diol. If low molecular weight diols containing ionic or potentially ionic groups are chosen, they may, for example, be used in an amount such that less than about 0.30 meq of —COOH is present per gram of polyurethane in the polyurethane dispersion. In at least certain exemplary and non-limiting embodiments, the low molecular weight diols containing ionic or potentially ionic groups are not used.

Reaction product (ii) may be chosen from at least one chain extender according to the formula:

H2N—R4-NH2

wherein R4 is chosen from alkylene or alkylene oxide radicals, said radicals not being substituted with ionic or potentially ionic groups.

Reaction product (ii) may optionally be chosen from alkylene diamines, such as hydrazine, ethylenediamine, propylenediamine, 1,4-butylenediamine and piperazine; and alkylene oxide diamines such as dipropylamine diethylene glycol (DPA-DEG available from Tomah Products, Milton, Wis.), 2-methyl-1,5-pentanediamine (Dytec A from DuPont), hexanediamine, isophoronediamine, and 4,4-methylenedi(cyclohexylamine), and the DPA-series of ether amines available from Tomah Products, Milton, Wis., including dipropylamine propylene glycol, dipropylamine dipropylene glycol, dipropylamine tripropylene glycol, dipropylamine poly(propylene glycol), dipropylamine ethylene glycol, dipropylamine poly(ethylene glycol), dipropylamine 1,3-propanediol, dipropylamine 2-methyl-1,3-propanediol, dipropylamine 1,4-butanediol, dipropylamine 1,3-butanediol, dipropylamine 1,6-hexanediol and dipropylamine cyclohexane-1,4-dimethanol, and mixtures thereof.

Reaction product (iii) may be chosen from at least one chain extender according to the formula:

H2N—R5-NH2

wherein R5 is chosen from alkylene radicals substituted with ionic or potentially ionic groups. In at least certain exemplary embodiments, the compounds may have an ionic or potentially ionic group and two isocyanate-reactive groups.

As used herein, ionic or potentially ionic groups may include groups comprising ternary or quaternary ammonium groups, groups convertible into such groups, carboxyl groups, carboxylate groups, sulphonic acid groups, and sulphonate groups. At least partial conversion of the groups convertible into salt groups of the type mentioned may take place before or during the mixing with water. Specific compounds include diaminosulphonates, such as for example the sodium salt of N-(2-aminoethyl)-2-aminoethanesulphonic acid (AAS) or the sodium salt of N-(2-aminoethyl)-2-aminopropionic acid.

In at least certain embodiments, R5 represents an alkylene radical substituted with sulphonic acid or sulphonate groups. By way of example only, the compound is chosen from sodium salts of N-(2-aminoethyl)-2-aminoethanesulphonic acid.

By way of non-limiting example, such latexes include, but are not limited to, aqueous polyurethane dispersions comprising a reaction product of a prepolymer comprising a dihydroxyl compound, a polyisocyanate, and a low molecular weight diol and at least two diamine compounds and wherein the composition is substantially free of triethanolamine stearate such as, for example, those sold under the BAYCUSAN® name by Bayer such as, for example, BAYCUSAN® C1000 (INCI name: Polyurethane-34), BAYCUSAN® C1001 (INCI name: Polyurethane-34), BAYCUSAN® C1003 (INCI name: Polyurethane-32), BAYCUSAN® C1004 (INCI name: Polyurethane-35) and BAYCUSAN® C1008 (INCI name: Polyurethane-48). In various exemplary embodiments, polyurethane latexes may be chosen from, but are not limited to, aqueous polyurethane dispersion of Isophthalic Acid/Adipic Acid/Hexylene Glycol/Neopentyl glycol/Dimethylolpropanoic Acid/Isophorone Diisocyanate copolymer (INCI name: Polyurethane-1, such as Luviset® P.U.R, BASF), aliphatic polyurethane and aliphatic polyester polyurethane (such as the Neorez® series, DSM, such as Neorez® R989, INCI name: Polycarbamyl Polyglycon Ester).

In at least certain embodiments, the simple latex polymers may be chosen from polyacrylic latex, polyacrylate latex, polystyrene latex, polyester latex, polyamide latex, polyurea latex, polyurethane latex, epoxy resin latex, cellulose-acrylate latex, and their copolymers.

### Hybrid Latexes

According to various exemplary embodiments of the disclosure, the compositions comprise at least one hybrid latex polymer. As used herein, the phrase “hybrid latex polymer” is meant to include multiphase particulate latex polymers comprising at least one supple phase and at least one rigid phase, such as, for example, core-shell and IPN polymers. In various embodiments according to the disclosure, the at least one hybrid latex polymer may be identified as polymer C.

In at least certain exemplary and non-limiting embodiments, the multiphase particles containing rigid and supple phases may optionally be film-forming, and may be capable of adhering to the substrate to which they are attached.

### Core Shell

In at least certain exemplary embodiments, polymer C may be chosen from an aqueous emulsion comprising a core-shell latex in which the core comprises a rigid polymer and the shell comprises a supple polymer. In various exemplary and non-limiting embodiments, the morphology of the core-shell type latex polymers may comprise shell portions completely surrounding the core, or may be of the core-shell type with a plurality of cores.

The supple polymer of polymer C may, in various embodiments, have a glass transition temperature (Tg) of less than or equal to about 60° C., such as, for example, from about −120° C. to about 60° C. In further exemplary embodiments, the glass transition temperature (Tg) may be less than or equal to about 45° C., such as, for example, from about −120° C. to about 45° C. In yet further exemplary embodiments, the glass transition temperature (Tg) may be less than or equal to about 30° C., such as, for example, from about −120° C. to about 30° C.

The supple polymer may, in various embodiments, be chosen from free radical polymers and polycondensates. By way of example only, the supple polymer may be chosen from poly(meth)acrylics, poly(meth)acylates, poly(meth)acylamides, polyurethanes, polyolefins, polyesters, polyvinyl ethers, and combinations thereof, such as, for example, those polymerized from the monomers described for simple latex polymers A and B herein.

The amorphous materials of the rigid polymer of polymer C may, in various embodiments, have a glass transition temperature (Tg) of greater than or equal to about 60° C., such as ranging from about 60° C. to about 200° C. In further exemplary embodiments, the glass transition temperature (Tg) may be greater than or equal to about 70° C., such as ranging from about 70° C. to about 200° C., or from about 70° C. to about 150° C. In yet further exemplary embodiments, the glass transition temperature (Tg) may be greater than or equal to about 90° C., such as ranging from about 90° C. to about 150° C.

The amorphous material of the rigid polymer of polymer C may, in various exemplary embodiments, be chosen from poly(meth)acrylic acid, poly(meth)acrylates, poly(meth)acrylamides, polyvinyls, polyvinyl esters, polyolfeins, polystyrenes, polyvinyl halides, polyvinylnitriles, polyurethanes, polyesters, polyamides, polycarbonates, polysulfones, polysulfonamides, polycyclics containing a carbon-based ring in the main chain, for instance polyphenylenes or polyoxyphenylenes, and combinations thereof. It may, for example, be polymerized from the monomers described for simple latex polymers A and B herein.

In various exemplary embodiments, the supple and rigid phases of the multiphase particles may comprise at least one free-radical polymer obtained by, or essentially obtained by, polymerization of monomers chosen from the group formed by (meth)acrylic acid esters, for instance alkyl (meth)acrylates, especially containing a C1-C8 alkyl group; vinyl esters of linear or branched carboxylic acids, such as vinyl acetate or vinyl stearate; styrene and its derivatives, such as chloromethylstyrene or α-methylstyrene; conjugated dienes, such as butadiene or isoprene; acrylamide, methacrylamide and acrylonitrile; vinyl chloride; and (meth)acrylic acid. The selection of monomers (nature and content), which may be a single monomer or a mixture of at least two monomers, of the supple polymer and of the amorphous material of the rigid phase, may optionally be determined by the glass transition temperature that it is desired to give to each polymer, which can be determined by those of skill in the art.

The polymers of the rigid and/or supple phases may be crosslinked with monomers containing at least two copolymerizable double bonds, chosen, for example, from conjugated dienes, such as butadiene or isoprene; allylic esters of α, β-unsaturated carboxylic acids, such as allyl acrylate or allyl methacrylate; allylic esters of α, β-unsaturated dicarboxylic acids, such as diallyl maleate; polyacrylics or polymethacrylics generally comprising at least two ethylenic unsaturations, such as ethylene glycol dimethacrylate, 1,3-butylene glycol dimethacrylate, 1,4-butanediol diacrylate or pentaerythritol tetraacrylate; polyvinyls such as divinylbenzene or trivinylbenzene; and polyallylics such as triallyl cyanurate.

Said chemical grafting allows, by the formation of covalent bonds, stable bonding of the rigid phase and the supple phase of the multiphase particles.

The supple polymer may be grafted onto the rigid polymer by means of a grafting monomer, said monomer possibly being a monomer containing several double bonds (ethylenic bonds), for example a monomer containing two ethylenic double bonds. The grafting monomer may be a conjugated diene such as those described above or an allylic ester (especially diester) of a, n-unsaturated dicarboxylic acids such as those described above (such as, for example, diallyl maleate) which contain two polymerizable functions (ethylenic double bond) of different reactivity: one of the polymerizable functions (ethylenic double bond) of the grafting monomer is polymerized with the polymer of the amorphous material of the rigid phase (rigid polymer) and the other polymerizable function (ethylenic double bond) of the same grafting monomer is polymerized with the supple polymer.

The multiphase particulate latex polymer C may be prepared using any commonly known methods. In at least one exemplary embodiment, it is prepared using at least one emulsion polymerization, in an essentially aqueous continuous phase, resulting in a stable dispersion in a physioligically acceptable medium.

The particles containing rigid and supple phases may have a size ranging from about 1 nm to about 10 mm, such as ranging from about 10 nm to about 1 mm, for example. The particle size may be optionally be measured using a Brookhaven BI-90 machine by the technique of light scattering, or with a Malvern Mastersizer 2000 granulometer, or alternatively by electron microscopy.

Typically, the rigid phase and the supple phase will be incompatible, i.e. they can be distinguished using the techniques that are well known to those skilled in the art, such as, for example, the technique of observation by electron microscopy or the measurement of several glass transitions of the particles by differential calorimetry. The multiphase particles are thus not homogeneous particles.

One exemplary and non-limiting example of preparation of multiphase particles includes a multi-stage emulsion polymerization, where the multiphase particles are prepared by consecutive series of polymerizations, with different types of monomers. In this procedure, the particles of a first family of monomers may be prepared in a separate step, or formed in situ by polymerization. Next, or at the same time, at least one other family of other monomers may be polymerized during at least one additional polymerization step. For example, as an exemplary polymer emulsion, first a core phase may be created using the above mentioned monomers (the first stage), followed by the creation of an outer shell phase using different ethylenically unsaturated monomers (the second stage). The particles thus formed have at least one internal structure, or core, and at least one external structure, or shell. As such, in at least certain embodiments of preparation, it is necessary for the structure of the external supple phase to be more supple than the structure of the internal rigid phase. The formation of a “multilayer” heterogeneous structure is thus possible. A wide variety of morphologies may flow therefrom, such as of the core-shell type, but also, for example, with fragmented inclusions of the rigid phase in the supple phase, all of which are intended for use according to various embodiments of the disclosure.

In various embodiments of the multiphase particles, the supple phase may be at least partly or may be predominantly external, and the rigid phase may be at least partly or may be predominantly internal, although this is not required.

Commerically available examples of such hybrid, multiphase particulate latex polymers include, but are not limited to, aqueous emulsion comprising a reaction product of a core acrylate and a shell acrylate, such as, for example, the core shell latex sold under the name of Acudyne DHR and Acudyne 1000, by Dow.

### IPN Latexes

In yet further exemplary embodiments, the at least one hybrid latex may be chosen from aqueous emulsions of interpenetrating polymer network (IPN) latexes, which are multi-modal particles that have at least two interlocking polymers, one soft and one hard, that are synthesized simultaneously or sequentially such that the two polymers are at least partially interlaced to form a network.

In at least certain, non-limiting exemplary embodiments, the IPN latexes may be chosen from those prepared as an aqueous emulsion, and having a particle ranging from about 50 nm to 100 nm. Exemplary IPN latexes may have a Tg ranging from about −50° C. to about 130° C., such as, for example, from about −45° C. to about 130° C.

Non-limiting examples of IPN latex include those described in the publication Solvent-free Urethane-acrylic Hybrid Polymers for Coating, E. Galgoci et. al, JCT Coatings Tech, 2 (13), 28-36 (February 2005), as well as in U.S. Pat. Nos. 4,644,030 and 5,173,526, incorporated herein.

For example, the IPN polyurethane/acrylic latex can be prepared following the procedure described in U.S. Pat. No. 5,173,526, incorporated herein:
a. form a dispersible polyurethane prepolymer with the terminal isocyanate containing carboxylic groups in water;
b. add a mixture of ethylenically unsaturated vinyl monomers to swell the prepolymer;
c. add an tertiary amine to the mixture of prepolymer/vinyl monomers to neutralize the carboxylic acid groups;
d. add radical initiator and the chain extender to the aqueous dispersion; and
e. heat the aqueous dispersion to start the polymerization process.

The prepolymer and the chain extender can be according to those described above for simple polyurethane latex polymers. The ethylenically unsaturated vinyl monomers can be according to those described above for simple acrylate latex polymers.

Commercially available examples of such hybrid, multiphase particulate latex polymers include, but are not limited to, aqueous emulsions comprising a reaction product of a polyurethane network intertwined with a reaction product of an acrylate network, such as, for example, the interpenetrating polymer network sold under the names of Hybridur 875, Hybridur 870, and Hybridur 880, by Air Products.

In various embodiments according to the disclosure, the interpenetrating polymer network latex may comprise both acrylate and polyurethane parts at the molecular level.

### Compositions

As described herein, exemplary compositions according to the disclosure may comprise at least one simple latex polymer, wherein the at least one latex polymer is a film-forming polymer. In further embodiments, the compositions may comprise at least two simple latex polymers, wherein at least one simple latex polymer is a film-forming polymer. According to various embodiments, each of the simple latex polymers is present in an amount ranging from about 0.05% to about 10% by weight, such as about 0.1% to about 7.5% by weight, such as about 0.25% to about 5% by weight, such as about 0.5% to about 2.5% by weight, or about 0.5% to about 1.5% by weight, relative to the weight of the composition, including all ranges and subranges there between. In other embodiments, each of the simple latex polymers is present in an amount ranging from about 1% to about 15% by weight, such as about 1% to about 12% by weight, such as about 1.2% to about 12% by weight, such as about 1.5% to about 10% by weight, or such as less than about 10% by weight, relative to the weight of the composition, including all ranges and subranges there between. In yet other embodiments, each of the simple latex polymers is present in an amount ranging from about 0.1% to about 2% by weight, such as about 0.15% to about 1.9% by weight, or such as about 0.18% to about 1.8% by weight, relative to the weight of the composition, including all ranges and subranges there between.

In certain embodiments, the simple latex polymers are present in an amount ranging from about 0.1% to about 30% by weight, such as about 0.1% to about 25% by weight, such as about 0.2% to about 20% by weight, such as about 0.2% to about 15% by weight, such as about 0.5% to about 10% by weight, such as about 1′)/0 to about 8% by weight, such as about 1% to about 5% by weight, such as about 1% to about 3% by weight, or such as below about 30% by weight, or such as about 25% by weight, or such as about 20% by weight, relative to the weight of the composition, including all ranges and subranges there between. By way of non-limiting example, the amount of simple latex polymers may be about 0.1%, about 0.5%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%, by weight, relative to the weight of the composition.

In yet further embodiments, the amount of simple latex polymers ranges up to about 30%, such as up to about 29%, such as up to about 28%, such as up to about 27%, such as up to about 26%, such as up to about 25%, such as up to about 24%, such as up to about 23%, such as up to about 22%, such as up to about 21%, such as up to about 20%, such as up to about 19%, up to about 18%, up to about 17%, up to about 16%, up to about 15%, up to about 14%, up to about 13%, up to about 12%, up to about 11%, up to about 10%, up to about 10%, up to about 9%, up to about 8%, up to about 7%, up to about 6%, up to about 5%, up to about 4%, up to about 3%, up to about 2%, or up to about 1%, each by weight, relative to the weight of the composition. In at least one exemplary embodiment, the amount of simple latex polymers is less than about 10% by weight, such as less than about 5% by weight, relative to the weight of the composition.

When more than one simple latex polymer is present, the amount is that of the combined amount of the simple latex polymers.

According to various embodiments of the disclosure comprising at least two simple latex polymers, the weight ratio of the at least two simple latex polymers, e.g. polymer A to polymer B, may range from about 10:1 to about 1:10, such as about 9:1 to about 1:9, about 8:1 to about 1:8, about 7:1 to about 1:7, about 6:1 to about 1:6, about 5:1 to about 1:5, about 4:1 to about 1:4, about 3:1 to about 1:3, or about 2:1 to about 1:2, including all ranges and subranges there between. It should be understood that when polymer A and/or polymer B comprise at least one simple latex film-forming polymer, the weight ratio includes the total amount of polymer A and/or polymer B.

According to various embodiments of the disclosure, the weight ratio of polymer A to polymer B is about 10:1, about 9:1, about 8:1, about 7:1, about 6:1, about 5:1, 4:1, about 3:1, about 2:1, about 1:1, about 1:2, about 1:3, about 1:4, about 1:5, about 1:6, about 1:7, about 1:8, about 1:9, or about 1:10.

In at least certain exemplary and non-limiting embodiments, when polymer A is chosen from simple latex polymers having a Young's modulus ranging from about 0.1 MPa to about 10 MPa, and a strain, under stress at 0.5 MPa, of at least about 1%; and polymer B is chosen from simple latex polymers having a Young's modulus ranging from about 10 MPa to about 6 GPa, and a strain, under stress at 0.5 MPa, of less than about 5%, different weight ratios of polymer A to polymer B may be chosen to correspond to different hair styling applications. By way of example only, a weight ratio of polymer A to polymer B ranging from about 1:10 to about 1:1 may, in some embodiments, provide a high level of style hold; a weight ratio of polymer A to polymer B ranging from about 5:1 to about 10:1 may, in some embodiments, provide a medium to high level of style hold; and a weight ratio of polymer A to polymer B ranging from about 3:1 to about 10:1 may, in some embodiments, provide a light to medium level of style hold.

In various embodiments according to the disclosure, the at least one hybrid latex polymer may be present in the composition in an amount ranging up to about 90% by weight of the composition, such as up to about 85%, such as up to about 80%, such as up to about 75%, such as up to about 70%, such as up to about 65%, such as up to about 60%, such as up to about 55%, up to about 50%, up to about 45%, up to about 40%, up to about 35%, up to about 30%, up to about 25%, up to about 20%, up to about 15%, up to about 10%, up to about 5%, or up to about 1%, by weight of the composition. For example, the at least one hybrid latex polymer may be present in an amount of about 0.1% to about 90%, about 0.5% to about 80%, about 1% to about 70%, about 2% to about 50%, about 2% to about 45%, about 5% to about 40%, or about 10% to about 30%, by weight of the composition.

According to certain exemplary and non-limiting embodiments, the hybrid latex polymer may be chosen such that it produces a film having a Young's modulus from about 0.1 MPa to about 6 GPa, and/or a strain, under stress at 0.5 MPa, that ranges up to about 300%.

In various embodiments, the hybrid latex polymers or polymer C of the disclosure may be chosen from those having a Young's modulus ranging from about 0.1 MPa to about 10 MPa, and a strain, under stress at 0.5 MPa, of at least about 1%. In other various embodiments, the hybrid latex polymers of the disclosure may be chosen from latex polymers having a Young's modulus ranging from about 10 MPa to about 6 GPa, and a strain, under stress at 0.5 MPa, of less than about 5%.

In at least certain exemplary and non-limiting embodiments, when the compositions of the disclosure employ a simple latex polymer chosen from Polymer A having a Young's modulus ranging from about 0.1 MPa to about 10 MPa, and a strain, under stress at 0.5 MPa, of at least about 1%, then it may be combined with at least one hybrid latex polymer having a Young's modulus ranging from about 10 MPa to about 6 GPa, and a strain, under stress at 0.5 MPa, of less than about 5%.

In other certain exemplary and non-limiting embodiments, when the compositions of the disclosure employ a simple latex polymer chosen from Polymer B having a Young's modulus ranging from about 10 MPa to about 6 GPa, and a strain, under stress at 0.5 MPa, of less than about 5%, then it may be combined with at least one hybrid latex polymer having a Young's modulus ranging from about 0.1 MPa to about 10 MPa, and a strain, under stress at 0.5 MPa, of at least about 1%

In yet other certain exemplary and non-limiting embodiments, when the compositions of the disclosure employ at least two simple latex polymers comprising Polymer A and Polymer B, then they may be combined with at least one hybrid latex polymer having a Young's modulus from about 0.1 MPa to about 6 GPa, and/or a strain, under stress at 0.5 MPa, that ranges up to about 300%.

In other embodiments, when the compositions of the disclosure employ at least two simple latex polymers comprising Polymer A, then they may be combined with at least one hybrid latex polymer having a Young's modulus ranging from about 10 MPa to about 6 GPa, and a strain, under stress at 0.5 MPa, of less than about 5%_{.}

In yet other embodiments, when the compositions of the disclosure employ at least two simple latex polymers comprising Polymer B, then they may be combined with at least one hybrid latex polymer having a Young's modulus ranging from about 0.1 MPa to about 10 MPa, and a strain, under stress at 0.5 MPa, of at least about 1%.

In addition to the combination of hybrid and simple latex polymers, wherein at least one simple latex polymer is a film-forming polymer, the compositions may further comprise at least one solvent. The at least one solvent may be chosen from water, at least one cosmetically acceptable organic solvent, or a mixture of water and at least one cosmetically acceptable organic solvent. Cosmetically acceptable organic solvents may, in various embodiments, be water-miscible, e.g. a mixture capable of forming at 25° C. a homogeneous mixture that is transparent, or substantially transparent, to the eye. For instance, cosmetically acceptable organic solvents may be chosen from lower monoalcohols, such as those containing from about 1 to 5 carbon atoms, for example ethanol and isopropanol; polyols, including glycols, such as those containing from about 2 to 8 carbon atoms, for example propylene glycol, ethylene glycol, 1,3-butylene glycol, dipropylene glycol, hexylene glycol, and glycerin; hydrocarbons, such as, for example, isododecane and mineral oil; and silicones, such as dimethicones, cyclomethicones, and cyclopentasiloxane; as well as mixtures thereof.

The at least one solvent may be present in an amount ranging up to about 95%, such as from about 1% to about 90%, from about 5% to about 80%, or from about 10% to about 60% by weight, relative to the total weight of the composition.

In at least certain exemplary embodiments, the latex polymer particles are not soluble in the solvent of the composition, and thus remain in particulate form even after evaporation of the solvent. For example, in embodiments where the composition comprises alcohol as a cosmetically acceptable organic solvent, the latex particles may remain in particulate form upon evaporation of the alcohol, such as once the composition is applied to a substrate.

Compositions according to various embodiments of the disclosure may further comprise additional components that are typically used in hair styling compositions. Such components are known to those of skill in the art, or are within the ability of those of skill in the art to determine depending on the particular application, such as, for example, the particular component and/or amount thereof. Such components include, but are not limited to, coalescing agents, plasticizers, and thickeners.

In various embodiments, the composition described herein may have a pH ranging from about 2 to about 9, such as about 3 to about 8, or about 4 to about 7.

In at least certain exemplary embodiments, the compositions are in the form of hair styling compositions, in any form, such as, for example, a gel, a cream, a foam, a lotion, an emulsion, or a liquid that may be sprayed onto or otherwise applied to the hair. In various embodiments, the composition may be provided in the form of a gel, a mousse, or a spray. In at least certain embodiments, the composition may be applied to the hair by first applying to the hands, and then contacting the hair with the hands; in other embodiments, the composition may be applied directly onto the hair, such as by spraying. The compositions may, in various embodiments, be applied to the hair as a leave-on treatment.

In various embodiments, the application of an external stimuli, such as heat, may be desirable as part of the hair styling process. By way of example only, before, during, or after the composition is applied to wet or dry hair, the hair may optionally be further treated with an external stimuli, for example with heat ranging from about 25° C. to about 250° C. In at least certain embodiments, the hair may also be shaped or positioned as desired while exposed to external stimuli, such as while heated or exposed to heat.

Professional and consumer heating tools can be used as a means to deliver heat or an elevated temperature to the hair. The heating tools can generate heat through electrical current or heating lamps. Depending upon the desired style, these tools include, but are not limited to, heaters, blow dryers, flat irons, hot combs, hot curler sets, steam pods, heated crimpers, heated lash curlers, heated wands/brushes, and hood driers or their combinations thereof.

As described, compositions according to the disclosure may impart a film on a substrate, such as on the hair or on the hand during or after application to the hair. A film formed by the composition may, surprisingly, be clean-feeling and not sticky, as with traditional hair styling compositions. Also surprisingly, the composition may impart a film on the hair that leaves the hair relatively natural and clean-feeling, yet has a flexible coating, leaving little to no residue, allows the hair to be bouncy and springy with little to no frizz or flaking, may impart relatively high definition with individualized curls, style control, volume, and shine, and/or may allow for relatively long-lasting hold and style memory. Furthermore, in at least certain embodiments according to the disclosure, the compositions are not sticky or tacky. A user of hair compositions according to various embodiments described herein may thus feel that the composition is not perceptible or is “invisible,” yet still effectively style and/or hold the hair. In at least certain embodiments according to the disclosure, the compositions may be quick-drying, which may allow drying and/or styling time to be reduced, as well as further improve ease of styling and curl retention. Additionally, the compositions may have effective hair styling and/or hold properties, even in conditions of high, or relatively high, humidity.

Furthermore, as described, compositions prepared according to various embodiments may provide for varying degrees of hold to be imparted to a hair style. By way of non-limiting example only, in order to obtain a spiky look to hair of a very short length, a high level of styling hold may be desirable. Or, as a further non-limiting example, in order to obtain a flowing look or to maintain hair curls for hair of medium length or longer length, a light to medium level of style hold may be desirable. By altering the weight ratio of the first and second polymers, it is possible to formulate compositions having high levels of style hold, medium to high levels of style hold, medium levels of style hold, or light to medium levels of style hold.

In at least certain embodiments, a film formed by the compositions described herein may be clear and/or stable. In such embodiments, phase separation and dramatic aggregation are minimized.

In addition, hair styled or treated with compositions according to the disclosure may, in at least certain exemplary embodiments, be hydrophobic, and/or may appear less frizzy and/or may be less prone to breakage, relative to hair subjected to the same conditions but not having been styled or treated with a composition according to the disclosure.

It should be noted, however, that compositions and films, as well as hair to which the composition or film has been applied, according to the disclosure may not have one or more of the herein-referenced properties, yet are intended to be within the scope of the disclosure.

Also disclosed herein are methods for styling the hair, said methods comprising applying a composition according to the disclosure to the hair, either before, during, or after styling the hair. One or more steps of treating the hair with an external stimuli, such as heat, before, during, or after the composition has been applied to the hair are also contemplated.

It is to be understood that both the foregoing description and the following Examples are exemplary and explanatory only, and are not to be interpreted as restrictive of the disclosure. Moreover, it should be understood that various features and/or characteristics of differing embodiments herein may be combined with one another. It is therefore to be understood that numerous modifications may be made to the illustrative embodiments and that other arrangements may be devised without departing from the scope of the disclosure. Other embodiments will be apparent to those skilled in the art from consideration of the disclosure and practice of the various exemplary embodiments disclosed herein.

It is also to be understood that, as used herein the terms “the,” “a,” or “an,” mean “at least one,” and should not be limited to “only one” unless explicitly indicated to the contrary. Thus, for example, the use of “a surfactant” is intended to mean at least one surfactant.

Unless otherwise indicated, all numbers used in the specification and claims are to be understood as being modified in all instances by the term “about,” whether or not so stated. The term “about” as it modifies numbers herein is meant to indicate a difference of 10% or less from the stated number, such as 9% or less, such as 8% or less, such as 7% or less, such as 6% or less, such as 5% or less, such as 4% or less, such as 3% or less, such as 2% or less, or such as 1% or less, in various exemplary embodiments. Thus, by way of example only, in one embodiment where “about” indicates a difference of 10% or less, the phrase “about 20%” is intended to encompass a range from 18%-22%. In another exemplary embodiment where “about” indicates a difference of 5% or less, the phrase “about 20%” is intended to encompass a range from 19%-21%. All such numbers within each specified range are hereby explicitly intended to be included in the disclosure.

It should also be understood that the precise numerical values used in the specification and claims form additional embodiments of the disclosure, and are intended to include any ranges which can be narrowed to any two end points disclosed within the exemplary ranges and values provided, as well as the specific end points themselves. Efforts have been made to ensure the accuracy of the numerical values disclosed herein. Any measured numerical value, however, can inherently contain certain errors resulting from the standard deviation found in its respective measuring technique.

It should be understood that compositions according to various embodiments of the disclosure form a film when applied to a substrate. However, the various properties of the film described herein are intended to include any film provided by compositions according to the disclosure, regardless of whether the film is attached or bonded to the substrate or not. By way of example only, once the compositions are applied to a substrate and a film is formed, the film may subsequently be removed in order to evaluate properties such as strain and Young's modulus.

### EXAMPLES

The following Examples are intended to be non-restrictive and explanatory only, with the scope of the invention being defined by the claims.

### Procedures

A. Procedures for Determination of Physical Properties of Films

Film Plating:

The latex film was obtained by allowing a 30 gram water solution containing 4 grams of the latex polymer(s) to dry slowly in a 100 mL PFA Petri dish (100 mm diameter×15 mm height) at room temperature for at least 3 days.

Film Measurement:

The latex film, with known dimensions (length, width, thickness), was mounted on the Q800 Dynamic Mechanical Analysis from TA Instrument, and tested in a DMA Control Force mode. The stress/strain test was obtained using the following procedure:

Preload force: 0.001 N

Isothermal: 25° C.

Soak time: 0.5 minutes

Force ramp rate: 0.5N/min to 18 N

The test ended when the sample broke, 18 N force was reached, or maximum displacement was achieved (25.5 mm).

From the stress/strain curve, the Young's Modulus was calculated as the slope of the linear portion at about 0.01% Strain to about 1% Strain. From the stress/strain curve, the % Strain at the stress of 0.5 MPa was also reported.

A high Young's Modulus demonstrates a hard film, while a lower Young's Modulus represents a more elastic film. A high Strain demonstrates a stretchy, elastic film, while a lower Strain represents a more brittle film.

B. Procedure for Determination of Mechanical Properties of Hair Treated with Latex Compositions

Hair Treatment:

A strip of regular bleached hair (from HIP, 1 cm in width, 16 cm long, about 2.0-2.5 g of hair) was treated with the latex solution (0.75 g of solution/g hair). The hair was combed through until the solution was uniformly distributed over the surface of the tress. The treated hair was allowed to dry overnight at room temperature.

Hair Measurement:

Three-point bending measurements were conducted using a texture analyzer (Model TA-XTPlus, Texture Technologies Corporation) equipped with a hair mounting accessory as described in J. Cosmet. Sci., 53, 345-362 (November/December 2002). The cantilever bending experiment consisted of the following sequence of steps: the hair tress was placed on a 2-point of 6 cm width, and the probe, representing the third point, came down at the middle of the hair tress and performed 10 cycles of 10-mm deformations of the hair tress. The testing protocol was:

Test mode=Compression

Pre-test speed=2 mm/sec

Test speed=2 mm/sec

Post-test speed=2 mm/sec

Target mode=Distance

Distance=10 mm

Count=10

Trigger type=Auto (Force)

Trigger force=1 g

After finishing 10 cycles of bending, a plot of force as a function of distance of 10 deformations was generated. From the plot, the maximum force in the first (F1) and the tenth (F10) deformation cycle was determined. The change from F1 to F10 was calculated from:

(F1−F10)/F1×100.

A high maximum force indicated that the hair was stiff and rigid, and a lower maximum force indicated that the hair was softer and more flexible.

Each experiment was run three times, and the results are reported from the average of the three experiments.

C. Procedure for Determination of Curl Retention in High Humidity of Hair Treated with Latex Compositions

Hair Treatment:

Regular bleached hair swatch (from HIP, 14.5 cm long, about 0.5 g) was treated with a solution of 2% latex polymers (0.5 g solution/g hair). The hair was combed until the solution was uniformly distributed over the hair swatch surface. The treated hair was then rolled onto a spiral rod (0.5 in diameter) and allowed to dry at room temperature overnight.

Curl Retention Measurement:

The coiled hair was removed from the rod and placed in the humidity chamber at 95% RH, 25° C. for 24 hours. The Curl Retention was calculated as:

(Lo−Lf)/(Lo−Li)×100

wherein Lo=fully extended hair length, Li=initial coiled hair length before humidity exposure, and Lf=final hair length after 24 hours exposure.

Compositions containing latex polymers were evaluated according to the methods described above. The weight of each latex polymer in the following examples is determined on a dry weight basis.

### Example 1

### Evaluation of Properties of Core Shell Latex and Simple Latex

Clear films are obtained from the combination of DAITOSOL 5000AD (INCI name: Acrylates Copolymer; polymer A1), BAYCUSAN C1001 (INCI name: Polyurethane-34; polymer A2), and ACUDYN DHR (INCI name: Acrylates/Hydroxyesters Acrylates Copolymer; polymer C), at various latex polymer ratios. Their physical properties are shown in Table 1 below.

TABLE 1

| | | Young's | Strain at 0.5 |
|---|---|---|---|
| | Component | Modulus | MPa stress |
| Sample | (A1:A2:C) | (MPa) | (%) |
| 1a | Polymer A1 only | 0.4 | >150 |
| 1b | Polymer A2 only | 4 | 27.54 |
| 1c | Polymer C only | 56 | 2.68 |
| 1d | 1:0:3 | 29 | 5.60 |
| 1e | 1:0:1 | 16 | 40.00 |
| 1f | 3:0:1 | 2 | >100 |
| 1g | 1:1:3 | 49 | 4.18 |
| 1h | 1:1:1 | 10 | 21.04 |
| 1i | 3:3:1 | 2 | 88.67 |

These results show that by varying the ratio of the combination of hybrid and simple latex polymers, it is possible to control the hardness and flexibility of films produced according to various embodiments of the disclosure.

### Example 2

### Evaluation of Properties of IPN Latex and Simple Latex

Clear films are obtained from the combination of DAITOSOL 5000AD (polymer A1), BAYCUSAN C1001 (polymer A2), and HYBRIDUR 875 (INCI name: Polyurethane-2 (and) Polymethymethacrylate; polymer C), at various latex polymer ratios. Their physical properties are shown in Table 2 below.

TABLE 2

| | | Young's | Strain at 0.5 |
|---|---|---|---|
| | Component | Modulus | MPa stress |
| Sample | (A1:A2:C) | (MPa) | (%) |
| 2a | Polymer A1 only | 0.4 | >150 |
| 2b | Polymer A2 only | 4 | 27.54 |
| 2c | Polymer C only | 418 | <0.1 |
| 2d | 0:1:3 | 351 | 0.16 |
| 2e | 0:1:1 | 109 | 0.50 |
| 2f | 0:3:1 | 26 | 3.51 |
| 2g | 1:1:1 | 185 | 0.37 |
| 2h | 1:1:3 | 21 | 4.31 |
| 2i | 3:3:1 | 4 | 39.93 |

These results show that by varying the ratio of the combination of hybrid and simple latex polymers, it is possible to control the hardness and flexibility of films produced according to various embodiments of the disclosure.

### Example 3

### Evaluation of Hair Treated with Core Shell Latex and Simple Latex

Hair tresses were treated with 2% solutions of DAITOSOL 5000AD (polymer A1), BAYCUSAN C1001 (polymer A2), and ACUDYN DHR (polymer C), at various latex polymer ratios. Their mechanical property is shown in Table 3 below.

TABLE 3

| | Component | F1 | Change in F |
|---|---|---|---|
| Sample | (A1:A2:C) | (g) | (%) |
| 3a | Polymer A1 only | 124 | 35 |
| 3b | Polymer A2 only | 399 | 27 |
| 3c | Polymer C only | 610 | 42 |
| 3d | 1:0:3 | 488 | 33 |
| 3e | 1:0:1 | 351 | 43 |
| 3f | 3:0:1 | 274 | 44 |
| 3g | 1:1:3 | 294 | 51 |
| 3h | 3:3:1 | 321 | 39 |
| 3i | Commercial 1* | 1835 | 76 |

*Main ingredients: VP/VA copolymer, polyquaternium-11, PEG 90 M, PEG-40 hydrogenated castor oil, acrylates/C10-30 alkyl acrylate crosspolymer, alcohol denatured.

These results show that hair tresses treated with various ratios of the combination of simple and hybrid latex polymers display a wide variety of rigidity, flexibility, stiffness, and softness. Compared to a commercial product (no latex), they show a significantly better styling durability due to the lower change in the maximum force after 10 cycles of deformation.

### Example 4

### Evaluation of Hair Treated with IPN Latex and Simple Latex

Hair tresses were treated with 2% solutions of DAITOSOL 5000AD (polymer A1), BAYCUSAN C1001 (polymer A2), and HYBRIDUR 875 (polymer C) at various latex polymer ratios. Their mechanical property is shown in Table 4 below.

TABLE 4

| | Component | F1 | Change in F |
|---|---|---|---|
| Sample | (A:B) | (g) | (%) |
| 4a | Polymer A1 only | 124 | 35 |
| 4b | Polymer A2 only | 399 | 27 |
| 4c | Polymer C only | 590 | 23 |
| 4d | 0:1:3 | 634 | 31 |
| 4e | 0:1:1 | 643 | 24 |
| 4f | 0:3:1 | 475 | 29 |
| 4g | 1:1:3 | 690 | 37 |
| 4h | 3:3:1 | 311 | 32 |
| 4i | Commercial 1* | 1835 | 76 |

*Main ingredients: VP/VA copolymer, polyquaternium-11, PEG 90 M, PEG-40 hydrogenated castor oil, acrylates/C10-30 alkyl acrylate crosspolymer, alcohol denatured.

The results in show that hair tresses treated with various ratios of the combination of hybrid and simple latex polymers display a wide variety of rigidity, flexibility, stiffness, and softness. Compared to a commercial product (no latex), they show a significantly better styling durability due to the lower change in the maximum force after 10 cycles of deformation.

### Example 5

### Evaluation of High Humidity Curl Retention of Hair Treated with Hybrid Latexes and Simple Latexes

Hair swatches were treated with 2% solutions of DAITOSOL 5000AD (polymer A1), BAYCUSAN C1001 (polymer A2), and HYBRIDUR 875 (polymer C) at various latex polymer ratios. The high humidity curl retention results are shown in Table 5 below.

TABLE 5

| | Component | Curl Retention |
|---|---|---|
| Sample | (A1:A2:C) | (%) |
| 5a | Polymer A1 only | 37 |
| 5b | Polymer A2 only | 39 |
| 5c | Polymer C only | 63 |
| 5d | 0:1:3 | 84 |
| 5e | 0:1:1 | 75 |
| 5f | 0:3:1 | 75 |
| 5g | 1:1:3 | 79 |
| 5h | 3:3:1 | 38 |

These results show that combination of simple and hybrid latex improves the curl retention, compared to the individual latexes.

### Example 6

### Evaluation of Effects of Concentration on Performance on Treated Hair—Core Shell Latex and Simple Latex

Regular bleached hair was treated with solutions of 1:1 and 1:1:1 ratios of DAITOSOL 5000AD (polymer A1), BAYCUSAN C1001 (polymer A2), and ACUDYN DHR (polymer C) at various latex polymer concentrations. The three-point bending test and the high humidity curl retention test was performed as described above. The results are shown in Tables 6A and 6B below.

TABLE 6A
Polymers C:A1

| | | F1 | Curl Retention |
|---|---|---|---|
| Sample | Concentration | (g) | (%) |
| 6a | 1% | 205 | 41 |
| 6b | 2% | 351 | 42 |
| 6c | 5% | 809 | 52 |
| 6d | 10% | 1391 | 76 |

TABLE 6B
Polymers C:A1:A2

| | | F1 | Curl Retention |
|---|---|---|---|
| Sample | Concentration | (g) | (%) |
| 6e | 1% | 212 | 45 |
| 6f | 2% | 187 | 35 |
| 6g | 5% | 885 | 55 |
| 6h | 10% | 1184 | 86 |
| 6i | Commercial 1* | 1835 | 42 |
| 6j | Commercial 2* | 4394 | 58 |

*Main ingredients: VP/VA copolymer, polyquaternium-11, PEG 90 M, PEG-40 hydrogenated castor oil, acrylates/C10-30 alkyl acrylate crosspolymer, alcohol denatured.
**Main ingredients: Water, Acrylates/steareth-20 methacrylate crosspolymer, polyquaternium-69, PVP, sorbitol and alcohol denatured.

These results demonstrate that as the concentration of the latexes increases, the hardness of the styled hair increases, as well as an increase in curl retention. It is noted that while having a wide range of hold, styled hair shows a significantly better hydrophobicity and humidity resistance compared to that treated with commercial (no latex) products.

### Example 7

### Evaluation of Effects of Concentration on Performance on Treated Hair—IPN Latex and Simple Latex

Regular bleached hair is treated with solutions of 1:1 and 1:1:1 ratios of DAITOSOL 5000AD (polymer A1), BAYCUSAN C1001 (polymer A2), and HYBRIDUR 875 (polymer C) at various latex polymer concentrations. The three-point bending test and the high humidity curl retention test are performed as described above. The results are shown in Tables 7A and 7B below.

TABLE 7A
Polymers C:A2

| | | F1 | Curl Retention |
|---|---|---|---|
| Sample | Concentration | (g) | (%) |
| 7a | 1% | 295 | 82 |
| 7b | 2% | 643 | 87 |
| 7c | 5% | 1256 | 100 |
| 7d | 10% | 2158 | 100 |

TABLE 7B
Polymers C:A2:A1

| | | F1 | Curl Retention |
|---|---|---|---|
| Sample | Concentration | (g) | (%) |
| 7e | 1% | 289 | 41 |
| 7f | 2% | 428 | 64 |
| 7g | 5% | 941 | 79 |
| 7h | 10% | 1487 | 95 |
| 7i | Commercial 1 | 1835 | 42 |
| 7j | Commercial 2 | 4394 | 58 |

These results demonstrate that as the concentration of the latexes increases, the hardness of the styled hair increases, as well as an increase in curl retention. It is noted that while having a wide range of hold, styled hair shows a significantly better hydrophobicity and humidity resistance compared to that treated with commercial (no latex) products.

What is claimed is:

1. A hair styling composition comprising:
(1) at least one simple latex polymer, wherein the at least one simple latex polymer is a film-forming polymer, and
(2) at least one hybrid latex polymer,
wherein the at least one simple latex polymer is present in an amount ranging from about 0.1% to about 30% by weight, relative to the weight of the composition, and
wherein the at least one hybrid latex polymer is present in an amount ranging up to about 90% by weight, relative to the weight of the composition.

2. The hair styling composition of claim 1, wherein the at least one simple latex polymer is chosen from simple acrylate latex polymers and simple polyurethane latex polymers.

3. The hair styling composition of claim 1, wherein the at least one simple latex polymer comprises dispersed particles in an aqueous dispersion medium.

4. The hair styling composition of claim 1, wherein the at least one simple latex polymer is present in an amount ranging from about 0.5% to about 10% by weight, relative to the weight of the composition.

5. The hair styling composition of claim 1, wherein the at least one simple latex polymer is present in an amount ranging from about 1% to about 5% by weight, relative to the weight of the composition.

6. The hair styling composition of claim 1, wherein the at least one simple latex polymer is present in an amount ranging from about 1% to about 3% by weight, relative to the weight of the composition.

7. The hair styling composition of claim 1, comprising at least two simple latex polymers, wherein at least one simple latex polymer is a film-forming polymer.

8. The hair styling composition of claim 7, wherein each of the at least two simple latex polymers is present in individual amounts ranging from about 0.05% to about 10% by weight, relative to the weight of the composition.

9. The hair styling composition of claim 7, wherein each of the at least two simple latex polymers is present in individual amounts ranging from about 1% to about 15% by weight, relative to the weight of the composition.

10. The composition of claim 7, wherein the at least two simple latex polymers are chosen from:
(1) polymer A, having a Young's modulus ranging from about 0.1 MPa to about 10 MPa, and a strain, under stress at 0.5 MPa, of at least about 1%; and
(2) polymer B, having a Young's modulus ranging from about 10 MPa to about 6 GPa, and a strain, under stress at 0.5 MPa, of less than about 5%.

11. The hair styling composition of claim 10, wherein the at least two simple latex polymers are, independently, chosen from acrylate latex polymers and polyurethane latex polymers.

12. The hair styling composition of claim 10, wherein the at least two simple latex polymers are present in the composition in a weight ratio of about 1:5 to about 5:1.

13. The hair styling composition of claim 10, wherein the at least two simple latex polymers are present in the composition in a weight ratio of about 1:3 to about 3:1.

14. The hair styling composition of claim 10, wherein the at least two simple latex polymers are present in the composition in a weight ratio of about 1:2 to about 2:1.

15. The hair styling composition of claim 10, wherein the at least two simple latex polymers are present in the composition in a weight ratio of about 1:1.

16. The hair styling composition of claim 10, wherein the weight ratio of polymer A to polymer B is about 1:10 to about 1:1.

17. The hair styling composition of claim 10, wherein the weight ratio of polymer A to polymer B is about 3:1 to about 10:1.

18. The hair styling composition of claim 10, wherein the weight ratio of polymer A to polymer B is about 5:1 to about 10:1.

19. The hair styling composition of claim 1, wherein the at least one hybrid latex polymer is present in an amount ranging from about 0.5% to about 80% by weight, relative to the weight of the composition.

20. The hair styling composition of claim 1, wherein the at least one hybrid latex polymer is chosen from core-shell and interpenetrating network polymers.

21. The hair styling composition of claim 20, wherein the at least one hybrid latex polymer is chosen from core-shell polymers comprising at least one rigid polymer core and at least one supple polymer shell.

22. The hair styling composition of claim 21, wherein the at least one rigid polymer comprises poly(meth)acrylic acid, poly(meth)acrylates, poly(meth)acrylamides, polyvinyls, polyvinyl esters, polyolfeins, polystyrenes, polyvinyl halides, polyvinylnitriles, polyurethanes, polyesters, polyamides, polycarbonates, polysulfones, polysulfonamides, polycyclics containing a carbon-based ring in the main chain, and combinations thereof, and the at least one supple polymer comprises poly(meth)acrylics, poly(meth)acylates, poly(meth)acylamides, polyurethanes, polyolefins, polyesters, polyvinyl ethers, and combinations thereof.

23. The hair styling composition of claim 1, further comprising at least one solvent.

24. A hair styling composition comprising:
(1) an aqueous dispersion comprising at least one hybrid latex polymer and at least one simple latex polymer, wherein the at least one simple latex polymer is a film-forming polymer, said aqueous dispersion comprising at least one simple polymer chosen from:
(a) polymer A, having a Young's modulus ranging from about 0.1 MPa to about 10 MPa, and a strain, under stress at 0.5 MPa, of at least about 1%; and
(b) polymer B, having a Young's modulus ranging from about 10 MPa to about 6 GPa, and a strain, under stress at 0.5 MPa, of less than about 5%;
wherein said at least one hybrid latex polymer is chosen from:
(c) polymer C, chosen from core shell and interpenetrating network polymers;
and
(2) at least one solvent;
wherein the at least one simple latex polymer is present in an amount of less than about 10% by weight, relative to the weight of the composition;
and wherein the at least one hybrid latex polymer is present in an amount ranging up to about 90% by weight, relative to the weight of the composition.

25. The hair styling composition of claim 24, comprising at least two simple latex polymers, wherein at least one simple latex polymer is a film-forming polymer.

26. A method of styling the hair, said method comprising applying a composition to the hair, said composition comprising:
(1) at least one simple latex polymer, wherein the at least one simple latex polymer is a film-forming polymer, and
(2) at least one hybrid latex polymer,
wherein the at least one simple latex polymer is present in an amount ranging from about 0.1% to about 30% by weight, relative to the weight of the composition; and
wherein the at least one hybrid latex polymer is present in an amount ranging up to about 90% by weight, relative to the weight of the composition.

27. The method according to claim 26, further comprising a step of treating the hair with heat at a temperature ranging from about 25° C. to about 250° C. before, during, or after the application of said composition.

28. The method of claim 26, wherein the hair styling composition comprises at least two simple latex polymers, wherein at least one simple latex polymer is a film-forming polymer.

29. A method of styling the hair, said method comprising a composition to the hair, said composition comprising:
(1) an aqueous dispersion comprising at least one hybrid latex polymer and at least one simple latex polymer, wherein the at least one simple latex polymer is a film-forming polymer, said aqueous dispersion comprising at least one simple polymer chosen from:
(a) at least one polymer A, having a Young's modulus ranging from about 0.1 MPa to about 10 MPa, and a strain, under stress at 0.5 MPa, of at least about 1%; and
(b) at least one polymer B, having a Young's modulus ranging from about 10 MPa to about 6 GPa, and a strain, under stress at 0.5 MPa, of less than about 5%;
wherein said at least one hybrid latex polymer is chosen from:
(c) polymer C, chosen from core shell and interpenetrating network polymers;
and
(2) at least one solvent;
wherein the at least one simple latex polymer is present in an amount of less than about 10% by weight, relative to the weight of the composition; and
wherein the at least one hybrid latex polymer is present in an amount ranging up to about 90% by weight, relative to the weight of the composition.

30. The method according to claim 29, further comprising a step of treating the hair with heat at a temperature ranging from about 25° C. to about 250° C. before, during, or after the application of said composition.

31. The method of claim 29, wherein the hair styling composition comprises at least two simple latex polymers, wherein at least one simple latex polymer is a film-forming polymer.
